(19) United States
(12) Patent Application Publication
(10) Pub. No.: US 2006/0089656 A1
(43) Pub. Date: Apr. 27, 2006

(54) Revision instruments

(75) Inventors: Randall Allard, Germantown, TN (US); Tom Francis, Cordova, TN (US); Jason Eckhardt, Memphis, TN (US); Chris Hughes, Cordova, TN (US); Lukas Eisermann, Memphis, TN (US); Peter McCombe, Brisbane (AU)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 10/971,852

(22) Filed: Oct. 22, 2004

(51) Int. Cl. A61F 2/34

(52) U.S. Cl. 606/99

(57) ABSTRACT

A system for removing an implant from between a pair of vertebral bodies comprises a removal shaft. The removal shaft includes a threaded shaft portion and is coupled to a spreader block. A connector body is threadedly coupled to the threaded shaft portion. The system further includes a first removal blade having a proximal end and a distal end. The proximal end is attached to the connector body and the distal end is attachable to the implant. A first spreader brace is attached to the spreader block. Rotation of the threaded shaft removes at least a portion of the implant from between the pair of vertebral bodies.

### BACKGROUND

In the treatment of diseases, injuries, or malformations affecting spinal movement and disc tissue, it has been common practice to remove a portion or all of a degenerated, ruptured, or otherwise failing disc. Following the loss or removal of disc or vertebral tissue, spinal implant devices have been implanted to promote fusion, restore motion to the treated area of the spine, or otherwise relieve pain in the spine. Occasionally after a spinal implant device has been installed and secured in the spine, revision procedures are required to modify or remove the device. Therefore, a method and apparatus are needed which allow safe and efficient removal of spinal implant devices.

### SUMMARY

In one embodiment of the present disclosure, a system for removing an implant from between a pair of vertebral bodies comprises a removal shaft. The removal shaft includes a threaded shaft portion and is coupled to a spreader block. A connector body is threadedly coupled to the threaded shaft portion. The system further includes a first removal blade having a proximal end and a distal end. The proximal end is attached to the connector body and the distal end is attachable to the implant. A first spreader brace is attached to the spreader block. Rotation of the threaded shaft removes at least a portion of the implant from between the pair of vertebral bodies.

In another embodiment of the present disclosure, a method is described for removing an implant from an intervertebral space between first and second vertebral bodies. The method comprises surgically exposing at least a portion of the implant. The implant includes a first endplate assembly having a first keel and a second endplate assembly having a second keel. The method further comprises inserting a first hooked osteotome around the first keel and extracting the first hooked osteotome and the first endplate assembly from the intervertebral space.

In another embodiment of the present disclosure, a revision method is described for removing an prosthesis from an intervertebral disc space. The method comprises attaching a first osteotome to a first endplate assembly of the prosthesis and attaching a second osteotome to a second endplate assembly of the prosthesis. The method further comprises coupling a tying member between the first and second osteotomes and applying a force to the tying member to withdraw both the first and second endplates from the intervertebral disc space.

In another embodiment of the present disclosure, a method for removing an intervertebral prosthesis from between first and second vertebral bodies comprises accessing the intervertebral prosthesis. The prosthesis has first and second endplate assemblies from which first and second keels, respectively, extend. The method further comprises inserting an osteotome between the first endplate assembly and the first vertebral body and forcing the first endplate assembly away from the first vertebral body. The method further comprises exposing a first aperture in the first keel and attaching a hooked probe to the first aperture. The hooked probe is pulled to separate and remove the first endplate from the first vertebral body.

In still another embodiment of the present disclosure, a system for removing an intervertebral implant is described. The implant includes first and second endplate assemblies and a core member extending between the first and second endplate assemblies. The system comprises a removal shaft having a threaded portion, the removal shaft coupled to a spreader block. The system further comprises a body housing threadedly coupled to the threaded portion and a removal blade having a proximal end and a distal end. The proximal end is attached to the body housing and the distal end is attachable to the implant. The system further comprises a first brace member attached to the spreader block and a second brace member attached to the spreader block. The first brace member and the second brace member adapted for holding the first endplate and the second endplate, respectively. Threading the threaded shaft into the body housing extracts the core member from between the first and second endplate assemblies.

### BRIEF DESCRIPTION OF THE DRAWINGS

FIG. 1 is a side view of vertebral column including a spinal implant device.

FIG. 2 is a perspective view of an intervertebral implant that may be revised with the instrumentation and methods provided by the disclosure.

FIG. 3 is a perspective view of a revision system.

FIGS. 4-7 are perspective views of components of the revision system of FIG. 3.

FIG. 8 is a perspective view of the revision system of FIG. 3.

FIGS. 9, 10a, 10b are alternative embodiments of a revision system according to the present disclosure.

FIG. 11 is another alternative embodiment of a revision system according to the present disclosure.

FIGS. 12-16 are another alternative embodiment of a revision system according to the present disclosure.

FIGS. 17-20 are side views of a vertebral column undergoing a revision procedure using a revision system according to another alternative embodiment of the present disclosure.

FIGS. 21-23 are perspective views of revision blades that may be used with any of the disclosed revision systems to loosen or clear space between an implant and bone.

FIGS. 24-25 are perspective views of additional removal instruments that may be used with any of the disclosed revision systems.

FIG. 26 is another alternative embodiment of a revision system according to the present disclosure.

FIG. 27 is another alternative embodiment of a revision system according to the present disclosure.

### DETAILED DESCRIPTION

The present disclosure relates generally to the field of orthopedic surgery, and more particularly to the instrumentation and techniques for spinal implant revision procedures. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

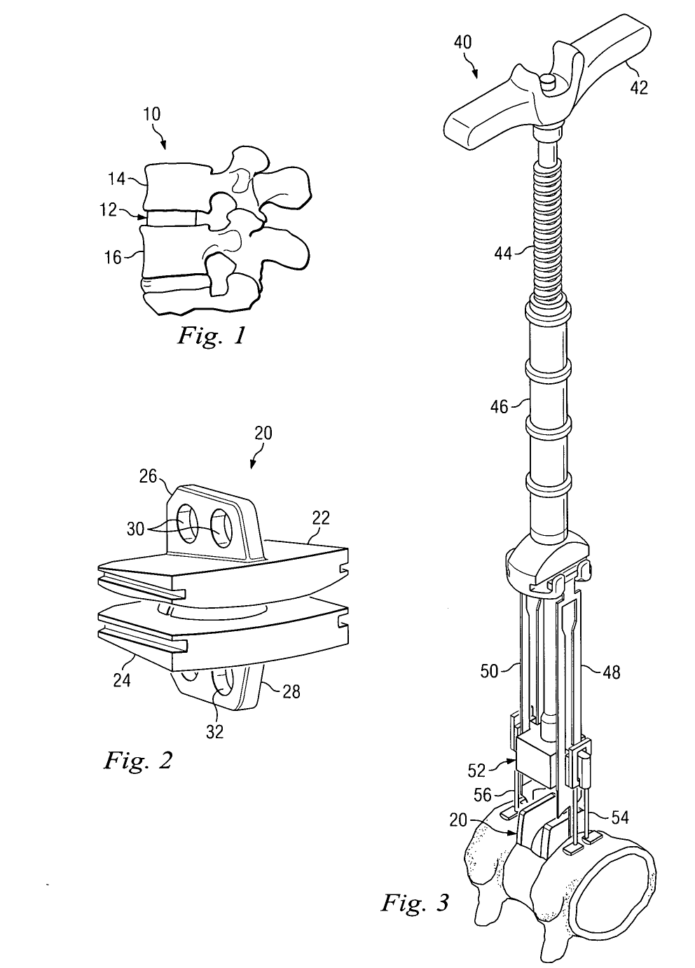

Referring to FIG. 1, the reference numeral 10 generally refers to a vertebral column with a spinal implant 12 extending between vertebral bodies 14, 16. The spinal implant 12 may be an intervertebral motion preserving disc such as the prostheses described in U.S. Pat. No. 6,740,118; and U.S. Patent Application Pub. Nos. 2004/0158328; 2004/0073312; and 2003/0204261, all assigned to SDGI Holdings, Inc. of Wilmington, Del. and incorporated by reference herein. The spinal implant 12 may, alternatively, be a prosthesis such as the ProDisc® System described in part by U.S. Pat. No. 5,314,477 and U.S. Patent Application Pub. No. 2004/0117022 (all incorporated by reference herein) and marketed by Spine Solutions, Inc. a subsidiary of Synthes, Inc. of Oberdorf, Switzerland. The spinal implant 12 may, alternatively, be a prosthesis such as the LINK® SB Charité™ Artificial Disc described in part by U.S. Pat. Nos. 4,759,766; 4,997,432; 5,401,269; 5,556,431; and 6,416,551 (all incorporated by reference herein) and marketed by Waldemar Link of Hamburg, Germany. In this disclosure, the spinal implant is primarily described as an intervertebral implant, but it is understood that the methods and apparatus of the invention may be applied to other types of spinal implants including intravertebral body and corpectomy implants.

Referring now to FIG. 2, the spinal implant 12 may be a prosthesis 20 having endplate assemblies 22, 24 from which keels, 26, 28, respectively, may extend. In this embodiment, the keel 26 includes apertures 30, and the keel 28 includes apertures 32. The prosthesis 20 is described more fully in U.S. Pat. No. 6,740,118, incorporated by reference above.

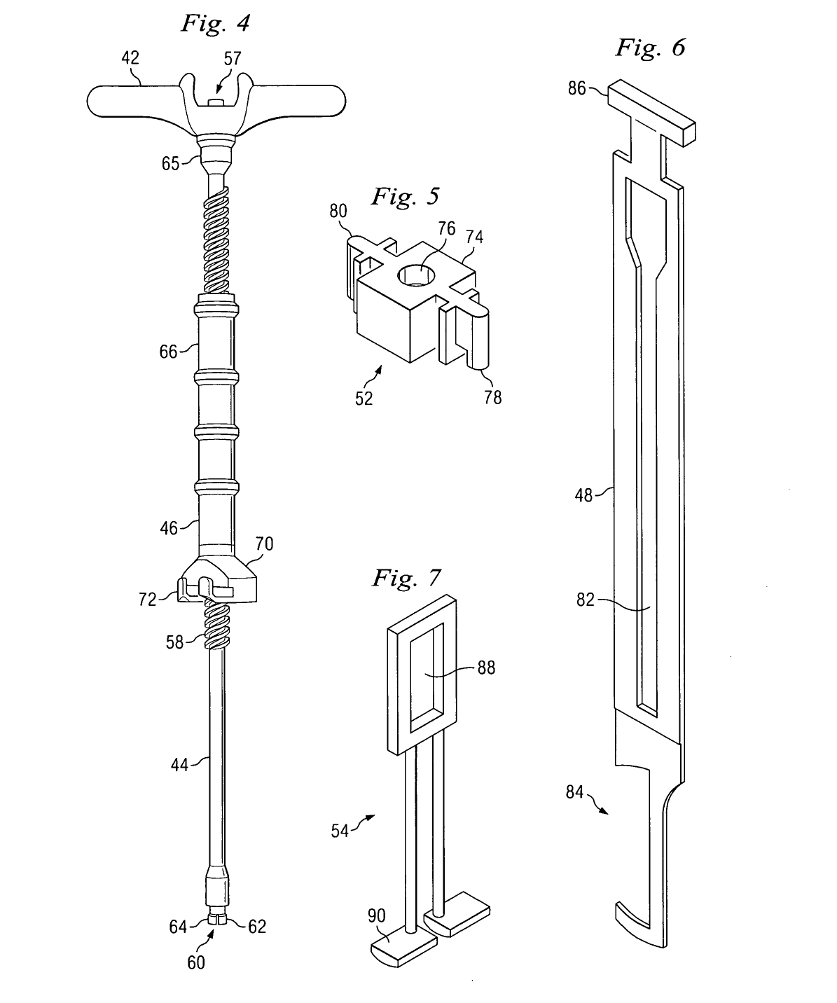

Referring now to FIGS. 3-7, a revision assembly 40 may be used to remove the prosthesis 20. In this embodiment, the revision assembly 40 includes a handle grip 42; a removal shaft 44; a connector body 46 which may function as a handle; a pair of osteotomes or removal blades 48, 50; a spreader block 52; and a pair of spreader braces 54, 56. The handle grip 42 may include a bore 57. The removal shaft 44 may include a threaded portion 58 for threadedly coupling to the connector body 46. The removal shaft 44 may further include a coupling end portion 60 having compliant splines 62 and a holding shaft 64 about which the spreader block 52 may pivot. The removal shaft 44 may further include an end portion 65 that may mate with the bore 57 of the handle grip 42. The connector body 46 may include a grip portion 66 and arms 70, 72. FIG. 5 depicts the spreader block 52 which may include a body 74 having a through bore 76. Supports 78, 80 may extend from opposite sides of the body 74. FIG. 6 depicts the removal blade 48 which may have an elongated slot 82, a hooked distal end 84, and an attachment end 86. The removal blade 50 may be substantially similar to the removal blade 48 and therefore is not described in detail. FIG. 7 depicts a spreader brace 54 which may include an attachment slot 88 and foot 90. The spreader brace 56 may be substantially similar to the spreader brace 54 and therefore is not described in detail.

As shown in FIG. 3, the revision assembly 40 may be assembled by inserting the end portion 65 into the bore 57 of the handle grip 42. The threaded portion 58 of the removal shaft 44 may be threaded into the connector body 46. The spreader block 52 may be attached to the coupling end portion 60 of the removal shaft 44 by compressing the compliant splines 62 and snapping the holding shaft 64 into the bore 76. The spreader block 52 may rotate about the holding shaft 64 while the uncompressed compliant splines 62 provide resistance to becoming pulled off. The spreader block 52 may be selected so that the distance between the supports 78, 80 is appropriately sized to span between the vertebral bodies 14, 16.

Removal blade 48 may be attached to the connector body 46 by connecting attachment end 86 to arm 70. Support 78 of the spreader block 52 may be positioned to extend through the elongated slot 82. Removal blade 50 may be similarly attached to arm 72 of the connector body 46. The connector body 46 may serve to tie the movement of the removal blades 48, 50 so that the blades may be removed simultaneously. Spreader brace 54 may be attached to the spreader block 52 by positioning the attachment slot 88 on the support 78. Spreader brace 56 may be attached to the support 80 in a similar manner. A tight tolerance between the spreader braces 54, 56 and the supports 78, 80, respectively, may help to maintain a firm connection. The spreader may include additional features such as notches, grooves, or ridges that further serve to hold the spreader braces 54, 56 firmly connected to the supports 78, 80, respectively.

In a surgical procedure to revise a prosthesis 20, the hooked distal end 84 of the removal blade 48 may be inserted between the implant 20 and the adjacent bone and hooked around the keel 26 of the prosthesis. Additional tools (not shown in this embodiment) may be used to drive the removal blade 48. Removal blade 50 may be attached to the keel 28 of the prosthesis 20 in a similar manner. As shown in FIG. 8, with the blades 48, 50 attached to the keels 26, 28, respectively, the threaded portion 58 of the removal shaft 44 may be forward threaded into the connector body 46, moving the spreader block 52, guided by the removal blades 48, 50, toward the prosthesis 20. As the spreader block 52 advances, the hooked distal ends of the removal blades 48, 50 may separate thus applying a distraction force to the adjacent vertebral endplates. Continued forward threading of the removal shaft 44 relative to the connector body 46 may bring the spreader braces 54, 56 into contact with the vertebral bodies 14, 16, respectively. Once contact is made, the continued forward threading of the removal shaft 44 may cause connector body 46 to pull the removal blades 48, 50 out of the intervertebral space, thereby extracting the prosthesis 20 from the vertebral column 10 with a predictable, controlled, slow, and smooth motion. As compared to other revision techniques that involve the application of outward force by the surgeon, the revision procedure using the revision assembly 40 may provide more protection to proximal tissues involved in anterior spinal surgery such as the aorta, vena cava, and iliac arteries and veins. The above described revision technique may also minimize damage to the vertebral bodies 14, 16 and maintain the integrity of the intervertebral disc space so as not to compromise the surgeon's ability to perform fusion or insertion of a different artificial disc.

Although the revision procedure described above has referenced the prosthesis 20, it is understood that alternative prostheses such as the ProDisc® System or the LINK® SB Charité™ Artificial Disc which may have keels, spikes, or other vertebral endplate protrusions may also be revised using substantially similar techniques to remove the endplate assemblies. The removal blades may be modified, as necessary, to connect to these alternative endplate protrusions.

The surgical procedure and instrumentation described above and depicted in FIGS. 3-8, may be modified in several ways based upon the preferences of the surgeon or the needs of the patient. For example, although the above described procedure is conducted with an anterior approach to the vertebral column 10, it is understood, that the methods taught by this disclosure may be used in alternative approaches such as lateral, anterior-lateral, oblique, or other desirable approaches.

The sequence of the revision steps described above may also be modified. For instance, in an alternative embodiment, the removal blades 48, 50 may be connected to the arms 70, 72, respectively, after the removal blades have first been attached to the keels of the implant. In another alternative embodiment, the spreader braces 54, 56 may be attached after the spreader block 52 has advanced toward the prosthesis 20 and is at a distance from the vertebral column 10 which is slightly larger than the length of the spreader braces 54, 56.

Rather than removing both endplate assemblies simultaneously, the revision system described above may be modified to employ a single removal blade rather than a pair, to remove one vertebral endplate assembly at a time. In still another alternative embodiment, the supports that extend from the body of the support block may be telescoping or adjustable to allow the spreader braces 54, 56 to be spaced apart as desired to maintain a secure interface with the surface of the vertebral bodies 14, 16.

Alternative removal blades, such as those described in detail below, may be used in addition to the removal blades 48 and 50 to assist in further separating the prosthesis from the vertebral bodies. Additionally, as described in further detail below, the hooked distal ends of the removal blades may be angled or shaped to more securely grasp the keels.

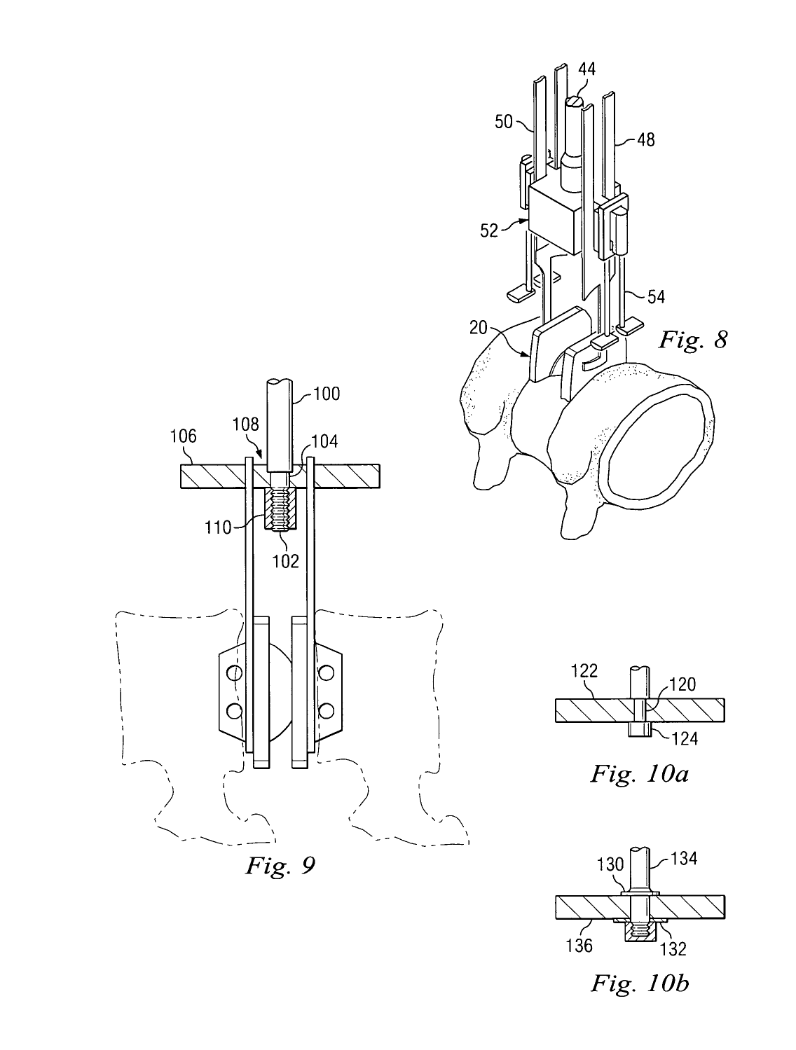

Alternative couplings between the removal shaft and the spreader block are also contemplated. For example, referring now to FIG. 9, a revision shaft 100 may include a threaded distal end 102 and a smooth portion 104. A spreader block 106 may include an aperture 108. The threaded distal end 102 may be inserted through the aperture 108 such that the smooth portion 104 is positioned in the aperture 108. A threaded spacer 110 may be attached to the threaded distal end 102 and advanced to a position such that the smooth portion 104 is permitted to pivot within the aperture 108. In another alternative, as shown in FIG. 10a, a revision shaft may include a distal end portion 120 about which a spreader block 122 may pivot. A stopper 124 may be positioned on the distal end portion 120 to prevent dislocation of the spreader block 122. In another alternative, as shown in FIG. 10b, washers 130, 132 may be positioned about a revision shaft 134, on opposite sides of a spreader block 136. The washers 130, 132 may create a low friction interface with the spreader block 136, allowing rotation of the spreader block 136 about the revision shaft 134.

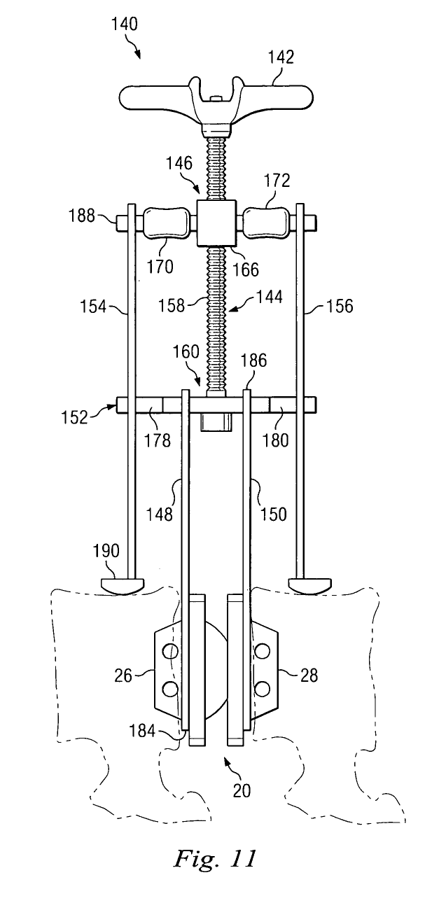

Referring now to FIG. 11, a revision assembly 140 is also contemplated. In this embodiment, the revision assembly 140 includes a handle grip 142; a removal shaft 144; a connector body 146; a pair of osteotomes or removal blades 148, 150; a spreader block 152; and a pair of spreader braces 154, 156. The removal shaft 144 may include a threaded portion 158 for threadedly coupling to the connector body 146. The removal shaft 144 may further include a coupling end portion 160 rotatably attached to the spreader block 152. The connector body 146 may include a grip portion 166 and arms 170, 172. Supports 178, 180 may extend from opposite sides of the spreader block 152. The removal blade 148 may have a hooked distal end 184 and an attachment end 186. The removal blade 150 is substantially similar to the removal blade 148 and therefore is not described in detail. A spreader brace 154 may include an attachment slot 188 and foot 190. Spreader brace 156 may be substantially similar to the spreader brace 154.

As shown in FIG. 11, the revision assembly 140 may be assembled by threading the threaded portion 158 of the removal shaft 144 into the connector body 146. The spreader block 152 may be attached to the coupling end portion 160 of the removal shaft 144 such that the spreader block 152 may pivot about the removal shaft 144. Removal blade 148 may be attached to the spreader block 152 by connecting attachment end 186 to support 178. Removal blade 150 may be similarly attached to support 180. Spreader brace 154 may be adjustably attached to the connector body 146 by threaded connection, ratchet connection, locking pin, or keyed connector, for example. Spreader brace 154 may also be movably connected to or guided by the support 178 of spreader block 152. Spreader brace 156 may be attached to the connector body 146 and the support 180 in a similar manner.

In a surgical procedure to revise a prosthesis 20, the hooked distal end 184 of the removal blade 148 may be inserted between the implant 20 and the adjacent bone and hooked around the keel 26 of the prosthesis. Removal blade 150 may be attached to the keel 28 of the prosthesis 20 in a similar manner. Spreader braces 154 and 156 may be positioned firmly against vertebral bodies 14, 16. As shown in FIG. 11, with the blades 148, 150 attached to the keels 26, 28, respectively, the threaded portion 158 of the removal shaft 144 may be reverse threaded out of the connector body 146, moving the spreader block 152 along the spreader braces 154, 156. The spreader braces 154, 156 may remain firmly held against the vertebral bodies 14, 16, respectively, by adjusting the connection between the braces and the connector body 146. The continued reverse threading of the removal shaft 144 may cause connector body 146 to pull the removal blades 148, 150 out of the intervertebral space, thereby extracting the prosthesis 20 from the vertebral column 10 with a predictable, controlled, slow, and smooth motion. As compared to other revision techniques that involve the application of outward force by the surgeon, the revision procedure using the revision assembly 140 may provide more protection to proximal tissues involved in anterior spinal surgery such as the aorta, vena cava, and iliac arteries and veins. The above described revision technique also minimizes damage to the vertebral bodies 14, 16 and maintains the integrity of the intervertebral disc space so as not to compromise the surgeon's ability to perform fusion or insertion of a different artificial disc.

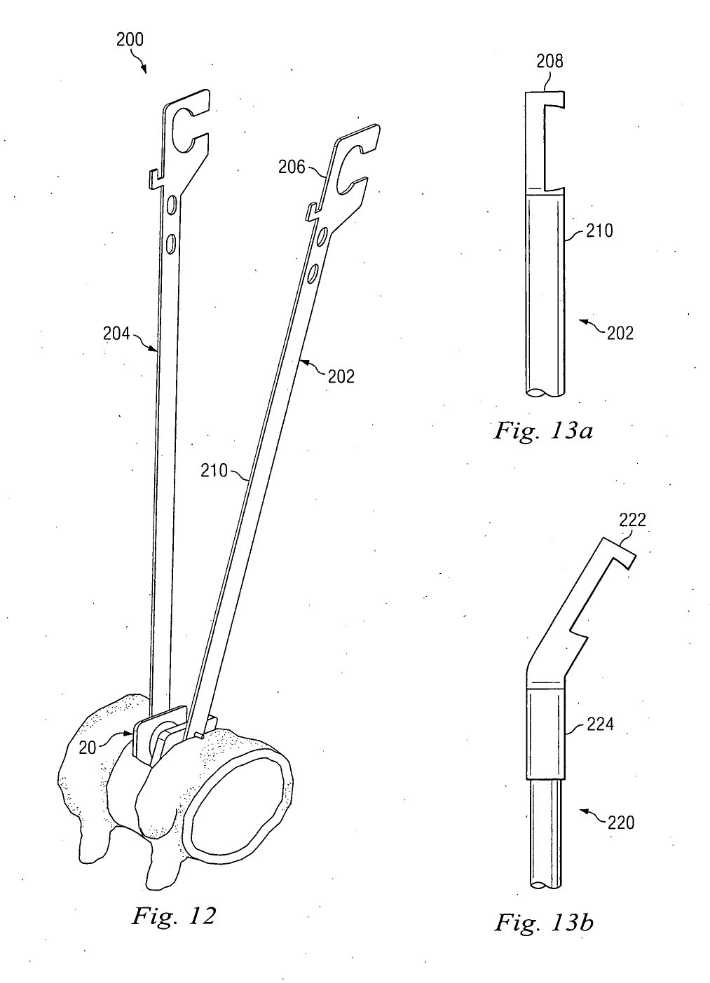

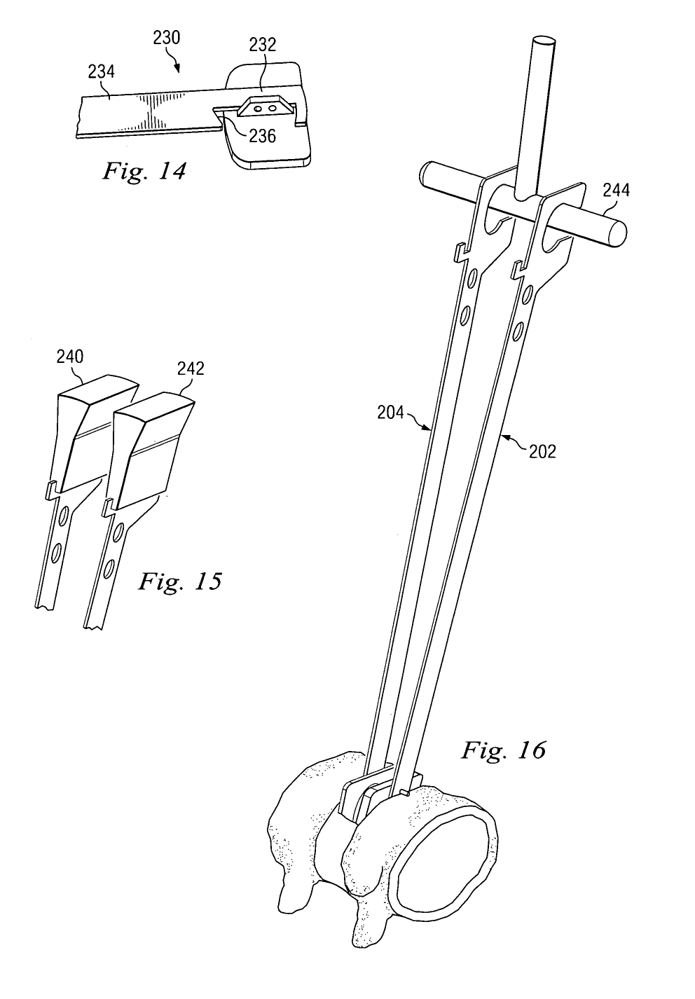

As shown in FIGS. 12-16, an alternative revision assembly 200 for a prosthesis 20 may include removal blades 202, 204. The removal blade 202 may include an attachment end 206, hooked distal end 208, and a shaft portion 210. As shown more clearly in FIG. 13a, the shaft portion 210 of the removal blade 202 may be in linear alignment with hooked distal end 208. In an alternative embodiment, as shown in FIG. 13b, a removal blade 220 may have a hooked distal end 222 and a shaft portion 224. In this embodiment, the hooked distal end 222 may be angled away from an axis defined by the shaft portion 224. In still another embodiment, as shown in FIG. 14, a removal blade 230 may have a hooked distal end 232 and a shaft portion 234. In this embodiment, the hooked distal end 232 may include a wedged lip 236 which may allow the hooked distal end 232 to catch and more securely hold a prosthesis keel.

The selection of removal blade may depend upon the lateral position of the prosthesis 20 and the ease of accessing the keels 26, 28. One of the removal blades 202, 220, 230 have all been described as having hooked distal ends, but, as will be described below, other types of removal blades or osteotomes may be used. The blades 202, 220, 230 may be inserted into the disc space and then rotated into position. The angled design of removal blade 220 may provide greater range of motion when rotating into place. The straighter osteotome 202 may allow for an easier pulling angle during removal. Each blade 202, 220, 230 may be marked, such as by laser etching, to indicate which side should face bone and which side should face the prosthesis 20. The hooked distal ends 208, 222, 232 may have tapered edges to ease insertion. The hooked distal ends 208, 222, 232 are sufficiently strong and wide to minimize the likelihood that the instruments will break off in the difficult to access posterior area of the intervertebral space.

As shown in FIGS. 15 and 16, the revision assembly 100 further includes impactor caps 240, 242; a T-handle 244; and a slap hammer (not shown). The impactor caps 240, 242 may allow for easy manipulation of the removal blades and may permit hammering from all directions if the implant space is limited.

In a surgical procedure to revise a prosthesis 20, the straight removal blades 202, 204 may be selected. The hooked distal end 208 of the removal blade 202 may be inserted between the implant 20 and the adjacent bone and hooked around the keel 26 of the prosthesis. Removal blade 204 may be attached to the keel 28 of the prosthesis 20 in a similar manner. To assist with the insertion of the removal blades 202, 204, impactor cap 240 may be connected to the attachment end 206 of the removal blade 202. Impactor cap 242 may be attached to the removal blade 204 in a similar manner. The impactor caps 240, 242 allow the surgeon to more easily grasp and manipulate the removal blades 102, 104 and also provide a larger surface area to hammer the blades into place between the implant 20 and the adjacent vertebral endplates. After the blades 202, 204 are in position around the keels 26, 28, respectively, of the implant 20, the impactor caps 240, 242 may be removed and the T-handle 244 may be attached to the removal blades 202, 204. The T-handle 244 may serve to tie the movement of the removal blades 202, 204 so that the blades may be withdrawn from the disc space together, rather than one at a time. With the T-handle 244 attached to the removal blades 202, 204, the slap hammer can be connected to the T-handle to pull both blades from the intervertebral space, thereby removing the prosthesis 20.

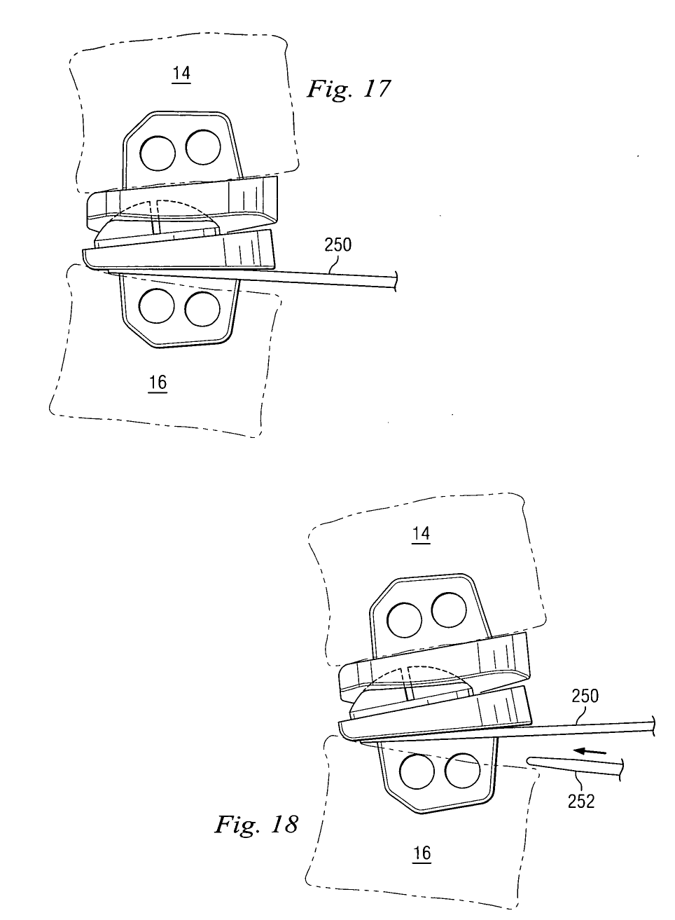

Referring now to FIGS. 17-20, an alternative procedure for removing the prosthesis 20 may include inserting a removal or revision blade 250 between the endplate assembly 24 and the adjacent endplate of vertebral body 16. FIGS. 21-25 depict several alternative removal or revision blades or instruments that may be used as the revision blade 250. For any given revision surgery, several of these revision blades may be included in the surgical instrumentation set available to the surgeon. The geometry and wedge thickness of the tips of the revision blades may be different to allow the surgeon more versatility during the procedure. As the revision procedure progresses, the surgeon may select the revision blades which may be most appropriate for a particular surgical approach or task.

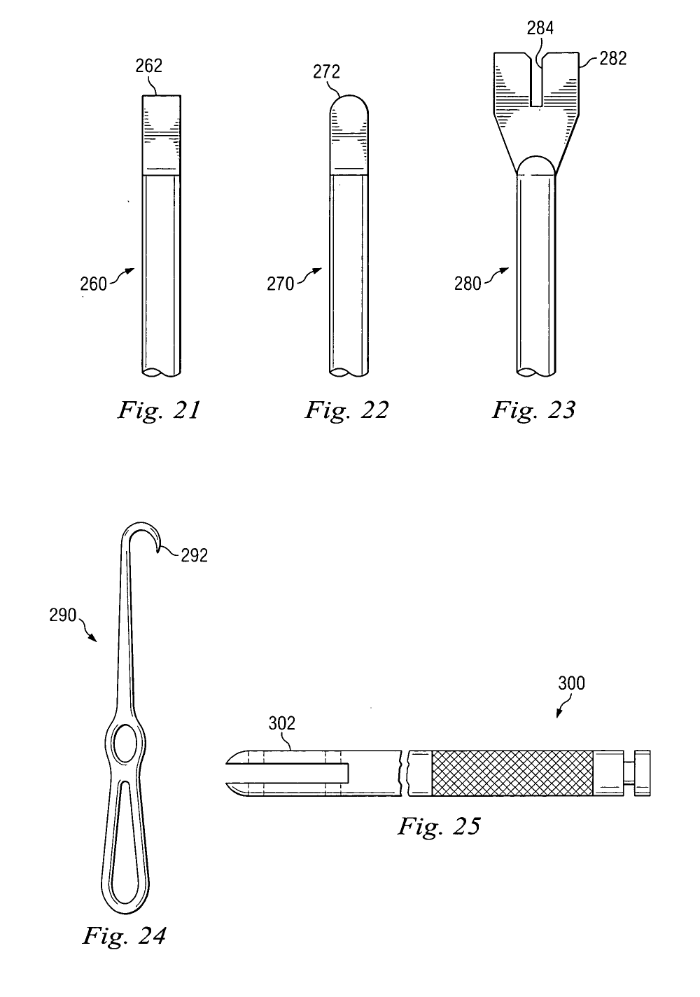

Referring to FIG. 21, a removal blade 260 includes a squared tip 262. Referring to FIG. 22, a removal blade 270 includes a rounded tip 272. Referring to FIG. 23, a removal blade 280 includes a broad forked tip 282. The tip 282 may include a notch 284 which is sized to fit around a keel on an endplate assembly. In use, the forked tip 282 may provide clearance capability from both sides of the keel. Referring to FIG. 24, a removal blade 290 may be a probe having a rounded hook tip 292. Referring to FIG. 25, a removal blade 300 includes a rounded forked tip 302.

Referring again to FIG. 17, the revision blade 250 may be inserted between the endplate assembly 24 and the adjacent endplate of the vertebral body 16. Hammers and impactions caps, such as those described above, may be used to position the blade 250. As the revision blade 250 is driven into place, the shape of the blade and/or a force applied by the surgeon may pry the endplate assembly 24 away from the endplate of vertebral body 16, creating clearance between the assembly and the endplate.

Referring to FIG. 18, a cutting instrument 252 such as a side cutting burr provided by Medtronic Midas Rex of Fort Worth, Tex. may be used to cut a passage through which the keel 28 may exit the vertebral body 16. This additional cutting may be particularly useful for late revision procedures that occur several months after the original implantation and after bony ingrowth and overgrowth has occurred.

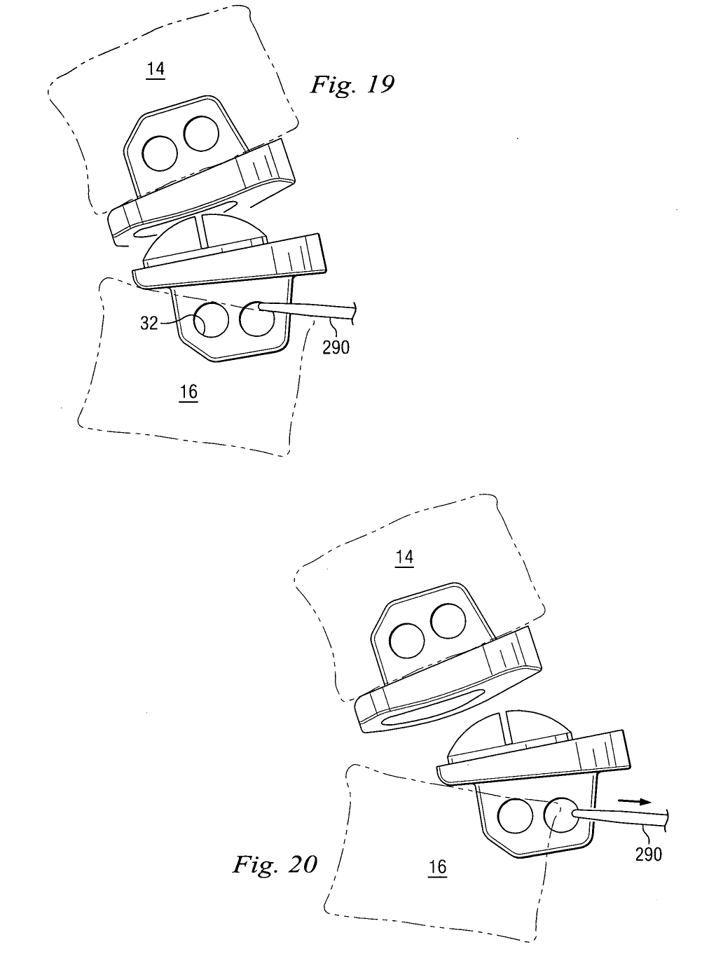

Referring to FIGS. 19-20, the revision blade 250 may separate the bone 16 and the endplate assembly 24 until at least a portion of the apertures 32 are accessible. The hooked osteotome or removal blade 290 may be inserted through one of the apertures 32 and may then be used to pull the endplate assembly 24 from the intervertebral disc space. A slap hammer or other removal instrument may be attached to the removal blade 290 to assist with the removal of the endplate assembly 24. This anterior fixation to the aperture 32 may be safer than alternative methods that require the placement of instrumentation in the posterior area of the intervertebral disc space because it minimizes the risk of breaking instrumentation in an area that is difficult to access. As it is pulled from the intervertebral space, the endplate assembly 24 may become separated from the endplate assembly 22. The steps described above may then be repeated to remove the endplate assembly 22 from the vertebral body 14.

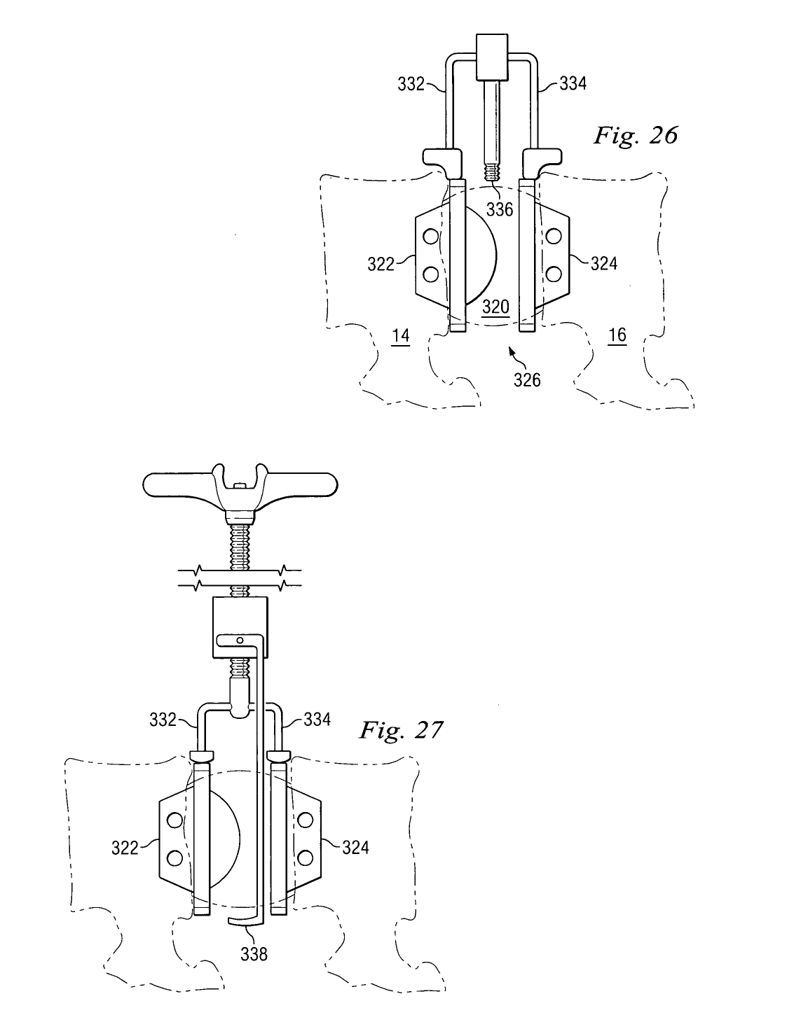

Referring now to FIG. 26, in still another alternative embodiment, the spinal implant 12 may be a prosthesis 320 having endplate assemblies 322, 324 between which a core member 326 may extend. The prosthesis 320 may, for example, be the ProDisc® System or the LINK® SB Charité™ Artificial Disc (incorporated by reference above). Prosthesis 320 may be revised as a single unit or may be dismantled one component at a time.

In this embodiment, a revision system 330 may be similar to the revision system 40 with the following modifications. The revision system 330 may include spreader braces 332, 334 configured to abut or attach to endplate assemblies 322, 324, respectively. The spreader braces 332, 334 may also abut or attach to the vertebral bodies, 14, 16, respectively to gain additional footing. A removal blade may extend into, hook around, or otherwise couple to the core member 326. FIG. 26 depicts a removal blade 336 that may tap and/or thread into the core member 326. FIG. 27 depicts a removal blade 338 that may hook around the core member 326. As the removal blade 336 or 338 is threadedly withdrawn from the intervertebral space, the core member 326 may be withdrawn from between the endplate assemblies 322, 324. With the spreader braces 332, 334 braced against the endplate assemblies 322, 324, the endplate assemblies may be held in place as the core member 326 is withdrawn. After the removal of the core member 326, a replacement core member may be inserted between the endplate assemblies 322, 324. If the entire prosthesis 320 is to be removed, the endplate assemblies 322, 324 may be removed, for example, using the procedure described above for FIGS. 17-20.

In an alternative embodiment, the entire three-piece prosthesis 320 may be revised by inserting a pair of removal blades through the core member 326 to hook to or otherwise connect to the vertebral endplate assemblies 322, 324. As the removal blades are withdrawn, the vertebral endplate assemblies 322, 324 and the core member 326 may be withdrawn from the intervertebral disc space. In still another alternative embodiment, the prosthesis 320 may be revised using instrumentation substantially similar to the revision system 200 modified to place a revision blade around or into the core member 326 for removal with a slap hammer.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications and alternative are intended to be included within the scope of this invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

What is claimed is:

1. A system for removing an implant from between a pair of vertebral bodies, the system comprising:
a removal shaft including a threaded shaft portion, the removal shaft coupled to a spreader block;
a connector body threadedly coupled to the threaded shaft portion;
a first removal blade having a proximal end and a distal end, wherein the proximal end is attached to the connector body and wherein the distal end is attachable to the implant; and
a first spreader brace attached to the spreader block,
wherein rotation of the threaded shaft removes at least a portion of the implant from between the pair of vertebral bodies.

2. The system of claim 1 wherein forward rotation of the threaded shaft portion removes the implant from between the pair of vertebral bodies.

3. The system of claim 1 wherein reverse rotation of the threaded shaft portion removes the implant from between the pair of vertebral bodies.

4. The system of claim 1 wherein the spreader block includes an adjustable arm for attaching to the first spreader brace.

5. The system of claim 4 wherein the adjustable arm is a telescoping arm.

6. The system of claim 1 wherein the first removal shaft comprises compliant splines adapted to permit a snap fit between the removal shaft and the spreader block.

7. The system of claim 1 further comprising a threaded spacer attached to the removal shaft to prevent separation of the spreader block from the removal shaft.

8. The system of claim 1 further comprising a stopper attached to the removal shaft to prevent separation of the spreader block from the removal shaft.

9. The system of claim 1 wherein the spreader block is rotatably coupled to the removal shaft.

10. The system of claim 1 wherein the first removal blade includes an alignment slot and the spreader block includes an alignment arm and wherein the alignment arm engages the alignment slot and is movable along the slot.

11. The system of claim 1 wherein the distal end of the first removal blade includes a hooked end portion adapted for grasping a keel located on the implant.

12. The system of claim 1 further including a second spreader brace attached to the spreader block.

13. The system of claim 12 wherein the first and second spreader braces are adapted to brace against the pair of vertebral bodies as the implant is removed from between the pair of vertebral bodies.

14. The system of claim 1 further including a second removal blade having a proximal end and a distal end, wherein the proximal end is attached to the connector body and wherein the distal end is attachable to the implant.

15. The system of claim 14 wherein rotation of the threaded shaft pulls the distal ends of both the first and second removal blades out from between the pair of vertebral bodies.

16. The system of claim 1 wherein the first spreader brace is movably attached to the spreader block and adjustably coupled to the connector body.

17. The system of claim 1 wherein the first spreader brace is fixedly attached to the spreader block.

18. The system of claim 1 further comprising a handle portion attached to the removal shaft.

19. The system of claim 1 wherein the first removal blade includes a straight distal end attachable to the implant.

20. The system of claim 1 wherein the first removal blade includes an angled distal end attachable to the implant.

21. The system of claim 1 wherein the spreader block is coupled to a non-threaded portion of the removal shaft.

22. The system of claim 1 wherein rotation of the threaded shaft removes an endplate assembly of the implant from between the pair of vertebral bodies.

23. The system of claim 1 wherein rotation of the threaded shaft removes a core member of the implant from between the pair of vertebral bodies.

24. A method for removing an implant from an intervertebral space between first and second vertebral bodies, the method comprising:
surgically exposing at least a portion of the implant, wherein the implant includes a first endplate assembly having a first keel and a second endplate assembly having a second keel;
inserting a first hooked osteotome around the first keel;
extracting the first hooked osteotome and the first endplate assembly from the intervertebral space.

25. The method of claim 24 wherein surgically exposing at least a portion of the implant includes exposing an anterior edge of the implant.

26. The method of claim 24 further comprising:
rotating a revision shaft to extract the first hooked osteotome and first endplate assembly from the intervertebral space.

27. The method of claim 26 further comprising:
attaching the first hooked osteotome to a revision body; and
threading the revision shaft into the revision body.

28. The method of claim 26 wherein rotating the revision shaft advances a spreader brace to engage the first vertebral body.

29. The method of claim 24 further comprising:
inserting a second hooked osteotome around the second keel;
extracting the second hooked osteotome simultaneously with the first hooked osteotome to extract both the first and second endplate assemblies from the intervertebral space.

30. An revision method for removing an prosthesis from an intervertebral disc space, the method comprising:
attaching a first osteotome to a first endplate assembly of the prosthesis;
attaching a second osteotome to a second endplate assembly of the prosthesis;
coupling a tying member between the first and second osteotomes;
applying a force to the tying member to withdraw both the first and second endplates from the intervertebral disc space.

31. The revision method of claim 30 further comprising attaching the first osteotome to a first keel on the first endplate assembly and attaching the second osteotome to a second keel on the second endplate assembly.

32. The revision method of claim 30 wherein applying the force includes advancing a threaded revision shaft into a handle body.

33. A system for revising an intervertebral implant disposed between a pair of vertebral endplates, the system comprising:
a first osteotome including a hooked end portion adapted for engaging a first endplate assembly of the intervertebral implant;
a second osteotome including a hooked end portion adapted for engaging a second endplate assembly of the intervertebral implant; and
a T-bar attached to and extending between the first and second osteotome,
wherein a force applied through the T-bar may dislocate the intervertebral implant from between the pair of vertebral endplates.

34. The system of claim 33 further comprising a slap hammer for applying the force through the T-bar.

35. The system of claim 33 further comprising a first impactor cap attached to the first osteotome.

36. The system of claim 33 wherein the hooked end portion of the first osteotome is straight.

37. The system of claim 33 wherein the hooked end portion of the first osteotome is angled.

38. A method for removing an intervertebral prosthesis from between first and second vertebral bodies, the method comprising:
accessing the intervertebral prosthesis, the prosthesis having first and second endplate assemblies from which first and second keels, respectively, extend;
inserting an osteotome between the first endplate assembly and the first vertebral body;
forcing the first endplate assembly away from the first vertebral body;
exposing a first aperture in the first keel;
attaching a hooked probe to the first aperture; and
pulling the hooked probe to separate and remove the first endplate from the first vertebral body.

39. The method of claim 38 further comprising:
milling the first vertebral body anterior to the first keel.

40. The method of claim 38 further comprising:
inserting an osteotome between the second endplate assembly and the second vertebral body;
forcing the second endplate assembly away from the second vertebral body;
exposing a second aperture in the second keel;
attaching the hooked probe to the second aperture; and
pulling the hooked probe to separate and remove the second endplate from the second vertebral body.

41. The method of claim 38 wherein the osteotome includes a square tip.

42. The method of claim 38 wherein the osteotome includes a rounded tip.

43. The method of claim 38 wherein the osteotome includes a forked tip.

44. The method of claim 43 further comprising:
creating clearance between the first endplate assembly and the first vertebral body on both sides of the keel using the forked tip of the osteotome.

45. A system for removing an intervertebral implant, the implant including first and second endplate assemblies and a core member extending between the first and second endplate assemblies, the system comprising:
a removal shaft having a threaded portion, the removal shaft coupled to a spreader block;
a first brace member attached to the spreader block, the first brace member adapted for holding the first endplate;
a second brace member attached to the spreader block, the second brace member adapted for holding the second endplate;
a body housing threadedly coupled to the threaded portion;
a removal blade having a proximal end and a distal end, wherein the proximal end is attached to the body housing and the distal end is attachable to the implant, and
wherein threading the threaded shaft into the body housing extracts the core member from between the first and second endplate assemblies.

46. The system of claim 45 wherein the distal end of the removal blade includes a hooked portion adapted to secure the core member.

47. The system of claim 45 wherein the distal end of the removal blade includes a threaded portion adapted to engage the core member.

48. The system of claim 45 further comprising:
an osteotome adapted for grasping the first endplate assembly.

49. The system of claim 45 further comprising:
an insertion instrument for inserting a replacement core member between the first and second endplate assemblies.

50. An revision method for removing a core member from between first and second endplate assemblies of an intervertebral prosthesis, the method comprising:
positioning a first brace on the first endplate assembly;
positioning a second brace on the second endplate assembly;
attaching a removal device to the core member; and
pushing the first and second braces against the first and second endplate assemblies while applying a counteracting force to the removal device to pull the core member from between the first and second endplate assemblies. 51. An revision method for removing an intervertebral implant from between first and second vertebral bodies, the method comprising:
positioning a first brace on the first vertebral body;
positioning a second brace on the second vertebral body;
attaching a removal device to the intervertebral implant; and
pushing the first and second braces against the first and second vertebral bodies while applying a counteracting force to the removal device to pull the intervertebral implant from between the first and second vertebral bodies.
